(19) United States
(12) Patent Application Publication
(10) Pub. No.: US 2021/0102899 A1
(43) Pub. Date: Apr. 8, 2021

(54) Chemically and Biologically Reactive Microplate Assembly and Manufacture Thereof for Raman Spectroscopy and Other Applications

(71) Applicants: Lloyd Ploof, Lee Center, NY (US); Jody Lee McRedmond, Ilion, NY (US); Thomas Joseph Basile, Clinton, NY (US)

(72) Inventors: Lloyd Ploof, Lee Center, NY (US); Jody Lee McRedmond, Ilion, NY (US); Thomas Joseph Basile, Clinton, NY (US)

(21) Appl. No.: 17/074,793

(22) Filed: Oct. 20, 2020

Related U.S. Application Data
(63) Continuation-in-part of application No. 16/156,612, filed on Oct. 10, 2018, now Pat. No. 10,829,846.
(60) Provisional application No. 63/061,392, filed on Aug. 5, 2020.
(60) Provisional application No. 62/723,635, filed on Aug. 28, 2018.

(51) Int. Cl. G01N 21/65; G01N 33/483

(52) U.S. Cl. CPC G01N 21/658; G01N 21/6458; G01N 33/483

(57) ABSTRACT

A microplate device, and the manufacture thereof, for detecting biologic, metallic, organic or inorganic substances using Raman Spectroscopy or equivalent analytical methods. The microplate has an array of wells that have metal, metal oxide, or a combination thereof on bottoms and/or sides of the wells. These may be coated with a substrate of metals and/or oxides that may physically or chemically react with or bind with a target substance or with another molecule, protein, or aptomer that then binds itself to the target substance. The metal(s) and/or metal oxides may react physically or chemically with a solution in which the target substance resides, making the target substance easier to detect.

This application claims priority under 35 U.S.C. 119(e) of Provisional Application Ser. No. 63/061392, filed Aug. 5, 2020, the disclosure of which is incorporated by reference herein. This application is also a Continuation-in-Part of co-pending U.S. patent application Ser. No. 16/156,612, filed Oct. 28, 2018 (now U.S. Pat. No. 10,829,846, Nov. 10, 2020), which claims priority under 35 U.S.C. 119(e) of Provisional Appin. Ser. No. 62/723,635, Aug. 28, 2018, the disclosure whereof is incorporated herein by reference.

### BACKGROUND OF THE INVENTION

Microplates are well-known tools for chemical and biochemical analysis, wherein multiple test samples are subjected to analysis for any of a variety of applications, e.g., medical diagnostics, food, beverage and cosmetics testing, environmental monitoring, manufacturing quality control, pharmaceutical research, and basic scientific research. Microplates of this general type are often constructed as a standard 96-well assay plate, in which an eight-by-twelve array of recesses or wells is formed. The wells each constitute a cell with a generally cylindrical cell wall and a horizontal cell floor. The microplates can be formed or molded of a neutral material, e.g., glass, a polymer, or in some cases a metal or silicon. The typical ninety-six well microplate is only about three and five-eighths inch by five inches, with the wells each occupying an area of about one-twentieth of a square inches, and typically with a shallow depth, so that the required amount of the target material and any reagent would be quite limited. The microplates are of generally standard sizes to they can be used in automated robotic testing equipment. There are other standard sizes, having more or fewer wells in each microplate. Moreover, due to the need to avoid contamination, these microplates or assay plates are often considered expendible, for one-time use only.

To date, microplates or assay plates have not been provided with a microstructured metallic substrate within the wells, i.e., on the floors or walls, to render them suitable for any of the varieties of Raman scattering or Raman spectroscopy that can be used for analysis of the test sample or targets material. Suitable microplates for other types of analysis have not been optimal. In particular, a microplate readily suitable for Surface Enhanced Raman Spectroscopy or SERS has not been available, or for other forms of analysis. The problem is not limited only to Raman spectroscopy.

In our earlier co-pending U.S. patent application Ser. No. 16/156,612, now U.S. Pat. No. 10,829,846, we disclosed a process for forming a substrate suitable for SERS, e.g., forming a framework of copper oxide dendrites on a copper substrate, which are then coated or plated with a noble metal, e.g., gold, silver or equivalent. This creates metal-coated dendrites with nano-structures, favorably in a range of 50 to 200 nm. This framework of noble-metal-coated copper-oxide dendrites is well suited for SERS analysis, and for many other related analytic techniques as well. We have found that placing such a framework of noble metal microstructures on the floor and/or walls of each of the wells of the microplate array provides an excellent test bed for automated analysis of small test quantities of target materials, using SERS or other forms of analysis.

The microplate can be created by a straightforward manufacturing process, and in this case can be especially adapted for Raman spectroscopy, including but not limited to surface-enhanced Raman spectroscopy for determining the make-up of an array of various test samples. Each of the wells (i.e., cups or cells) contains a small test sample of a substance. With the microplate being dimensioned as a standard size (e.g., a regular 12×8 array of wells) the microplate permits a SERS (or other) analysis to be done automatically and in a predetermined order on each of the 96 samples to be tested. In our process, there is a layer of copper in each well, which can be deposited galvanically, electrolytically, or electrolessly, or by vapor or ion deposition. This material is subjected to a treatment, which may involve a known oxidizer, to form copper oxide, which is in the form of microscopic CuO dendrites in the range of between about 0.001 nanometer to 100 nanometers. These dendrites may be in the form of needles, fern-like structures, of fan-shaped structures, and with the dendrites present over a wide range of sizes. Then the cupric oxide dendrites are coated are coated with another metal, in this case with a noble metal or monetary metal, such as gold or silver, or another equivalent metal such as Pa, Pt, or in some cases Sn, Ni, Cu, Rh or Zn, depending on the needs of the Raman spectroscopy process and the test or target material. Thereafter the metal-coated cupric oxide dendrites are cleaned and rinsed.

The nano-structured dendrites, covered with a thin coating of noble metal upon the copper-oxide dendrites, provides a superior response for SERS analysis of the test samples.

### SUMMARY OF THE INVENTION

In view of the need for new and improved ways for employing analytic technologies with high repeatability and accuracy, it is an important object of this invention to offer an economical means to create a microplate or similar arrayed test tray, coated with an appropriate microstructure of metal or metals, and/or metal oxides, to make possible an enhanced analysis that can be carried out on a routine basis.

It is another object to provide a microplate with an array of wells or test cells, in which bottoms and/or sides of the wells are given a substrate of metal and/or metal oxides, such that at the bottom and/or side of each well there exists a metal or metal oxide layer of any of the following: zinc, cadmium, nickel, lithium, silicon, gold, silver, palladium, platinum, rhodium, tin, iron, indium, copper or such other metal that may be applied as a metallic layer by electrolytic plating, electrodes plating, vacuum deposition, plasma deposition, flame spraying, immersion plating, or any other suitable method for depositing an adherent metallic layer to the bottom and/or sides of the well. The metal and/or metal oxide can exist just at the bottom or base of the well, or in some cases just at the side wall of the well. The wells are typically round, i.e., cylindrical, but can be of other geometries, as needed.

Conventionally, microplates are formed of a suitable solid material with the wells molded in. Often, the material is a molded plastic resin, such a polystyrene, but may be another suitable material such as acrylic, polypropylene, glass, silicon, or any other material compatible with the target material and the analysis to be carried out.

Most currently available microplates can be considered to be an array of mini-test tubes, simply holding the samples to be analyzed without reacting with them. In some cases, porous or semipros membranes have been added, gels with metal nano-particles have been placed in the bottoms of wells, or electrodes installed for luminance testing. In some cases, magnetic material has been included for the magnetic separation of certain biologic materials.

However, conventional microplates or variations that have been proposed cannot meet current and future technological demands. There is an unaddressed need for microplates that have surfaces that contact the test sample substances and can react with the substances in a way that aids in the analysis and assay during the testing procedure. One example of such procedure is Surface Enhanced Raman Spectroscopy, where the metallic microstructures create a plasmon that affects the spectrum for the sample and aids in identifying components in the sample. Other examples of such procedures are Tip Enhanced Raman Spectroscopy, Laser Desorption Ionization Spectrometry, X-Ray Photoelectron Spectroscopy, and X-Ray Fluorescence Microscopy. This is not an exhaustive list of the possible procedures. Particularly with Raman Spectroscopy, a modified metal surface aids in the creation of a plasmon that is essential in analysis. Certain metallic surfaces, or metaloxide surfaces, or some combinations of metals and metal oxides, are reactive with the substance of interest, or reactive with something to which the substance is attached, the reaction being physical or chemical to the subject substance of interest. The results of the physical or chemical reaction can be essential to an accurate and repeatable analysis.

A multiple-well microplate can be manufactured from a flat piece of suitable material in which the wells are molded or machined. This material may be PVC, ABS, acrylic, metal, Teflon, silicon, rubber, stainless steel, glass, glass fibers, or any other suitable material in a thickness of 0.01 inch to 1.5 inches, but preferably between about 0.10 to 0.50 inches. In this case, an array of through-holes are formed in the material, and another flat plate is attached, that plate being made of any of the above-mentioned materials. That flat plate may also be provided with metallized and treated disks at locations that are in register with the through-holes when the two plates are cemented or joined together. This forms a multiple well plate with an array of wells. These materials may be the same as is frequently used for printed circuit boards, as these are well suited to have metal and metal oxide layers deposited onto them. The microplate may have printed conductive strips connecting to some or all of the metallized disks.

### BRIEF DESCRIPTION OF THE DRAWING

FIG. 1 is a plan view of a multi-well plate or microplate of this invention, having an array of generally cylindrical wells (in this example ninety-eight), in which metal and/or metal oxide is deposited in bottoms and/or sides of each well.

FIG. 1A is a cross-section thereof.

FIG. 2 is a plan view of another multi-well microplate of this invention, of similar design to the embodiment of FIG. 1, except here having the metallic features formed on a separate plate joined to the main microplate which has an array of through-holes bored throught it.

FIGS. 2A and 2B are sectional assembly views of the flat bottom plate and bottom piece thereof.

FIG. 3 is a perspective view of a microplate of this invention formed of a durable synthetic plastic resin,

FIG. 4 is another perspective view thereof showing the general depth of the wells or cups.

FIG. 5 is a perspective view of another microplate of this invention, here with the top plate with an array of through-holes bored therein and next to it the bottom plate having bottom disks of suitable metal nanostructures deposited or plated thereon.

FIG. 6 is a top perspective of the assembled microplate of this embodiment.

FIG. 7 shows a related embodiment including metallized printed conductive strips.

### DETAILED DESCRIPTION OF PREFERRED EMBODIMENTS

In our earlier-filed U.S. patent application Ser. No. 16/156,612, filed Oct. 10, 2018, (Pub. No. US2020-0071812), we described the technology for producing a substrate that provides superior performance for use in surface-enhanced Raman spectroscopy, or SERS, by applying a suitable oxidizing agent to a copper metal substrate to produce cupric oxide dendrites, which form in a wide range of sizes extending well down into the nanoscale range, and then coating these copper oxide dendrites with another metal, preferably a monetary metal, such as gold. The resulting nano-structures of a monetary metal layer on the copper oxide nanostructures can be used to physically or chemically react with a test sample for Surface Enhanced Raman Spectroscopy. This material may also have applications with other related Raman techniques as well as in electronics, magnetics, batteries, solar cells, and others.

Many microplates of this general type have been produced, with an array of test wells (e.g., eight by twelve) or cups supported on or molded or bored into a main plate of a suitable material. In our invention the bores have the above-described nano-structure of metal-coated copper-oxide dendrides deposited onto the side walls and/or the bottoms of the wells.

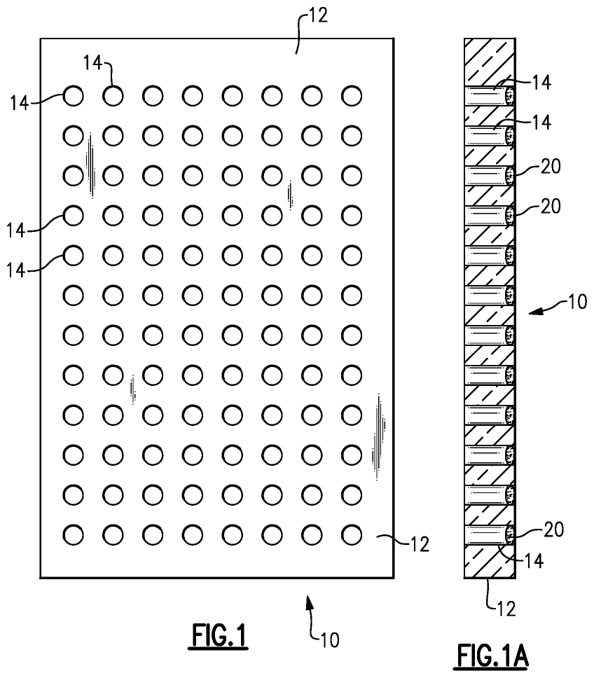

A microplate, microwell plate, or multiwell is a flat plate with multiple “wells” used as small test tubes. When constructed according to the present invention, the microplate can be constructed much like a standard microplate for research and clinical diagnostic testing, and one embodiment is shown in FIGS. 1 and 1A. Here a microplate 10 is formed as a thickness or plate 12 of solid material with an array of molded cups or wells 14. This can be formed of any of a number of synthetic resin materials, such as polystyrene, but may be other suitable material such as acrylic or polypropylene, as well as glass, silicon, and many other materials as may be suitable for the manufacture and compatible with the analysis carried out with these microplates. The microplate 10 here satisfies the above-identified need for surfaces inside the wells that are reactive to and aid in the analysis and assay during such procedures as Surface Enhanced Raman Spectroscopy, Tip Enhanced Raman Spectroscopy, Laser Desorption Ionization Spectrometry, X-Ray Photoelectron Spectroscopy, and X-Ray Fluorescence Microscopy, among others. In this case a surface treatment 20 of metallized copper oxide dendrides is placed at the bottom or floor, and optionally also along some part of the sides of the wells 14, as illustrated in FIG. 1A. As seen in this view, the covered bottom of each well 14 are typically flat, although there may be zones of spherical concave surfaces, on which the surface treatment 20 is placed. This construction facilitates use of the device in Raman Spectroscopy. Typically, target test substances are placed into the respective wells 14, where the modified metal surface of a surface treatment 20 aids in the forming plasmons that affect the stimulated emissions from the test substance under radiation impinging on it from a laser or other source. For other related analytic techniques, the metallic surface, or metal oxide surface, or any combination of metals and metal oxides, may be reactive with the substance of interest, or reactive with something that is attached, whether chemically or physically, to the substance of interest. In that way the inner surface of the wells can facilitate accurate and repeatable analysis.

The surface treatment 20 within the wells 14 may be a coating achieved by electroplating, electrodes plating, immersion plating, physical vapor deposition, flame spraying, or any other suitable means to produce a suitable adherent film on the bottom and/or sides of the wells 14 of a microplate 10.

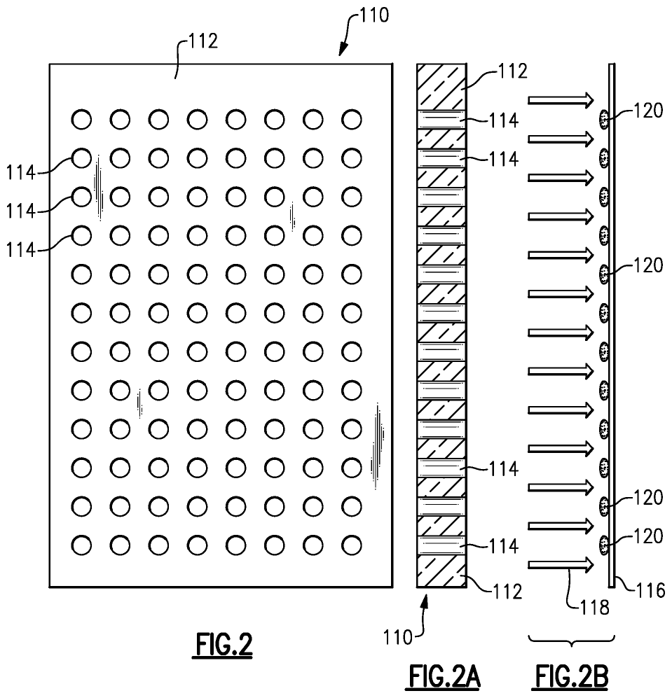

Another embodiment 110 of the microplate of this invention, as shown in FIG. 2, FIG. 2A and FIG. 2B, comprises an assembly of a rectangular plate 112 of material of suitable composition and thickness which has formed in it an array of through holes 114. This rectangular plate 112 is adhesively bound to a flat bottom piece 116 of a material similar to that of the plate 112, and this contains an array of disks 120 of any of the aforementioned metals and/or their oxides, or having any combination of any of these metals or oxides attached to its surface. The bottom piece 116 may favorably be formed of circuit board material, for example. The disks 120 are in register with the through holes 114 in the plate 112, and the bottom piece 116 may be welded, cemented, or otherwise bonded to the under surface of the rectangular plate 112, for example with double-sided tape 118 or a suitable adhesive. The resulting multi-well microplate has the metal(s) and/or oxides exposed at the bottom of the respective through holes 114, where they will contact test substances place in the through holes.

Other embodiments of this invention may also be achieved by incorporating the flat lower bottom piece 116 of plastic resin or circuit board material into the multi-well microplate during a molding process, such that the metallized disks 120 on bottom of the wells of the microplate are exposed. Bonding of the flat metal-containing piece and the side-wall piece would then be achieved.

A possible alternative construction could have the entire upper surface of the bottom piece 116 covered with the nano-structure metallic dendrites, but the described embodiment with the metallized dendrites limited to disks 120 is preferred as much less material would be needed.

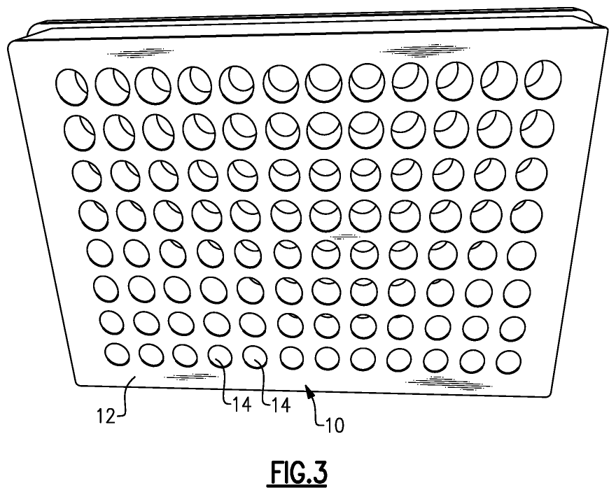

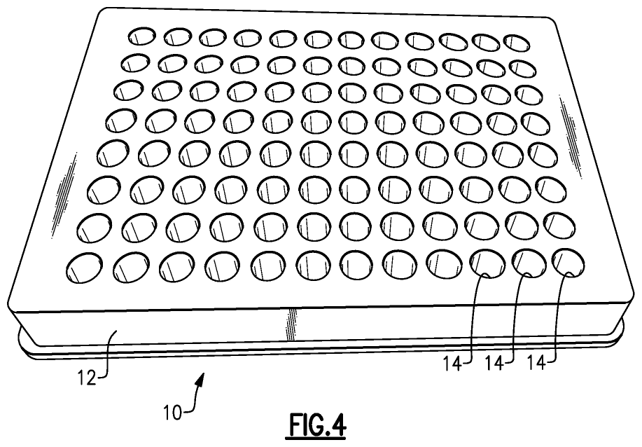

A practical version of the microplate of FIGS. 1 and 1A is presented in perspective in FIGS. 3 and 4, where the wells or cups 14 are molded of a rigid or semi-rigid plastic resin and incorporated into the flat plate portion 12 of the microplate 10. Here the flat plate 12 is formed hollow except for the cups or wells 14, making the microplate lighter and facilitating stacking and storage. The metallized copper oxide surface treatment 20 within each of the wells or cups is obscured in these views.

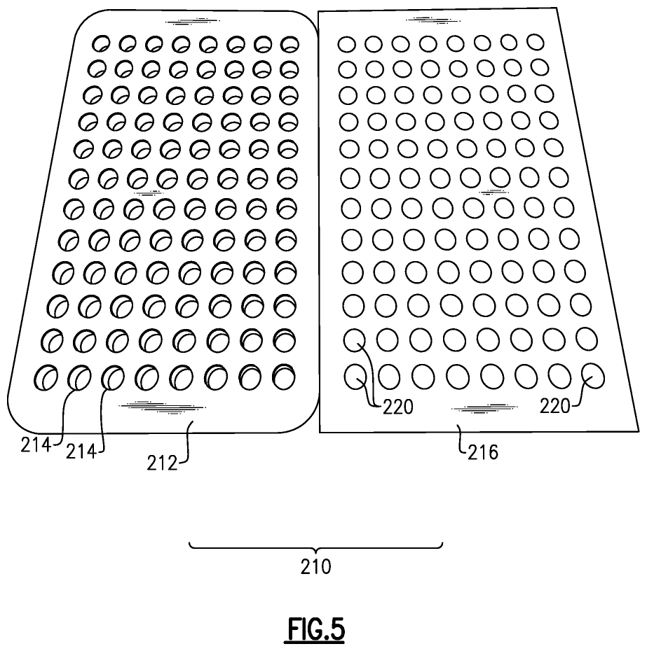

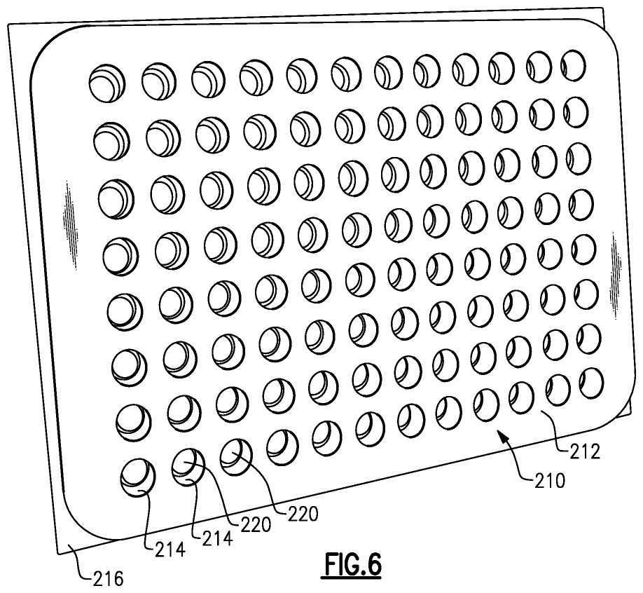

Another practical version of an embodiment, here of the microplate of FIGS. 2, 2A, and 1B is presented in perspective as microplate 210 in FIGS. 5 and 6. Here the two components are shown side by side, with an upper rectangular plate 212 on the left, with a regular array of through-holes 214, and a flat bottom piece 216 on the right, with the disks 120 of metallized copper oxide dendrite microstructures arrayed so as to be in register with the respective through holes 214. FIG. 6 shows the two components, that is, the upper rectangular plate 212 with through holes 214 assembled onto the flat bottom plate 216. Here the disks 220 are visible in the through-holes 114. In some cases, the sides of the through holes 214 could be provided with the substrate of metallized nano-structure dendrites prior to joining the upper and lower components.

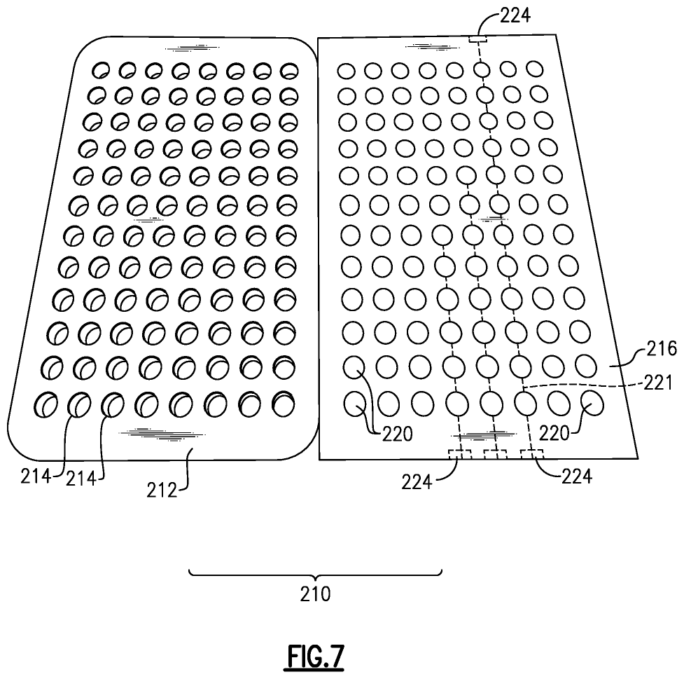

FIG. 7 shows a version of the microplate of FIGS. 5 and 6, except to illustrate that printed conductive metallized strips 221 (shown here as broken lines) can extend to and between some or all of the metallized copper oxide dendrite disks 220. The material in the wells can be energized through conductive vias. These conductive strips 221 may terminate at conductive pads 224 at edges of the bottom plate 216. The printed strips 221 can be on either the upper or lower surface of the bottom plate 216, or both. Current or a static or variable voltage, AC or DC, or pulsed current or any type of energy may be applied to some or all of the disks 220, as may be prescribed for a given type of analysis. The pattern of the conductive printed strips can have any desired configuration.

Many possible variations and re-configurations of microplates can incorporate the features and principles of the present invention, as defined in the appended claims.

What is claimed is:

1. Microplate assembly comprising
a plate dimensioned with a predetermined length and width;
a plurality of wells in said plate and arranged in a predetermined pattern thereon, each well having a top open at an upper side of said plate, a side wall and a floor;
a substrate applied on one or both of the side wall and floor of at least some of said wells, said substrate having a multiplicity of nano-scale dendrites each having a metallized surface configured to be in direct contact with test sample material when placed into the respective well.

2. Microplate assembly according to claim 1 wherein said plate is formed of a synthetic resin and said plurality of wells are molded into said plate, and said plate is formed of a synthetic resin and said plurality of wells are molded into said plate.

3. Microplate assembly according to claim 1 wherein said plate includes an upper plate portion in which are formed a plurality of through-holes at predetermined locations thereon; a lower plate portion adapted to be adhered against said upper plate portion and at least coextensive therewith, and with said lower plate portion having said substrate formed and disposed as disks at locations configured to be in register with respective ones of the through holes of said upper plate portion.

4. Microplate assembly according to claim 1 wherein said substrate is formed as dendrites of copper oxide, and said metallized surface is a noble metal deposited onto said dendrites.

5. Microplate assembly according to claim 1 wherein said nano-scale dendrites are effective to react physically with said test sample material.

6. Microplate assembly according to claim 1 wherein said nano-scale dendrites are effective to create plasmons that affect a spectroscopy spectrum for the test materials.

7. Microplate assembly according to claim 1 wherein said nano-scale dendrites are effective to react chemically with said test sample material.

8. Microplate assembly according to claim 1 wherein said predetermined pattern of wells is a regular geometric array such that the microplate assembly is configured for automated robotic testing.

9. Microplate assembly according to claim 3, with said lower plate portion having printed conductive metallized strips thereon extending to and between some or all of said disks.

10. Microplate assembly according to claim 9, wherein said conductive strips terminate at conductive pads at one or more edges of the lower plate portion.
